(19) United States
(12) Patent Application Publication
(10) Pub. No.: US 2002/0160998 A1
(43) Pub. Date: Oct. 31, 2002

(54) Pharmaceutical compositions and methods for use

(76) Inventor: Thomas Jeffrey Clark, High Point, NC (US)

Correspondence Address: CARL B. MASSEY, JR., WOMBLE CARLYLE SANDRIDGE & RICE, PLLC, POST OFFICE BOX 7037, ATLANTA, GA, 30357-0037, US

(21) Appl. No.: 09/845,470

(22) Filed: Apr. 30, 2001

(51) Int. Cl. A61K 31/55; A61K 31/5377; A61K 31/4748; A61K 31/427; A61K 31/422; A61K 31/4178

(52) U.S. Cl. 514/214.03; 514/235.8; 514/305; 514/397; 514/365; 514/374; 514/381; 540/582; 544/132; 546/133

(57) ABSTRACT

The present invention relates to aryl olefinic azacyclic compounds and aryl acetylenic azacylic compounds. The present invention relates in particular to five-membered heteroaromatic olefinic azacyclic compounds and five-membered heteroaromatic acetylenic azacylic compounds, including isoxazolyl olefinic cycloalkylamines and isoxazolyl acetylenic cycloalkylamines.

### BACKGROUND OF THE INVENTION

The present invention relates to pharmaceutical compositions, particularly pharmaceutical compositions incorporating compounds that are capable of affecting nicotinic cholinergic receptors. More particularly, the present invention relates to compounds capable of activating nicotinic cholinergic receptors, for example, as agonists of specific nicotinic receptor subtypes. The present invention also relates to methods for treating a wide variety of conditions and disorders, particularly conditions and disorders associated with dysfunction of the central and autonomic nervous systems.

Nicotine has been proposed to have a number of pharmacological effects. See, for example, Pullan et al., N. Engl. J. Med. 330:811 (1994). Certain of those effects may be related to effects upon neurotransmitter release. See, for example, Sjak-shie et al., Brain Res. 624:295 (1993), where neuroprotective effects of nicotine are proposed. Release of acetylcholine and dopamine by neurons upon administration of nicotine has been reported by Rowell et al., J. Neurochem. 43:1593 (1984); Rapier et al., J. Neurochem. 50:1123 (1988); Sandor et al., Brain Res. 567:313 (1991) and Vizi, Br. J. Pharmacol. 47:765 (1973). Release of norepinephrine by neurons upon administration of nicotine has been reported by Hall et al., Biochem. Pharmacol. 21:1829 (1972). Release of serotonin by neurons upon administration of nicotine has been reported by Hery et al., Arch. Int. Pharmacodyn. Ther. 296:91 (1977). Release of glutamate by neurons upon administration of nicotine has been reported by Toth et al., Neurochem Res. 17:265 (1992). Confirmatory reports and additional recent studies have included the modulation in the Central Nervous System (CNS) of glutamate, nitric oxide, GABA, takykinins, cytokines and peptides (reviewed in Brioni et al., Adv. Pharmacol. 37:153 (1997)). In addition, nicotine reportedly potentiates the pharmacological behavior of certain pharmaceutical compositions used for the treatment of certain disorders. See, for example, Sanberg et al., Pharmacol. Biochem. & Behavior 46:303 (1993), Harsing et al., J. Neurochem. 59:48 (1993) and Hughes, Proceedings from Intl. Symp. Nic. S40 (1994). Furthermore, various other beneficial pharmacological effects of nicotine have been proposed. See, for example, Decina et al., Biol. Psychiatry 28:502 (1990); Wagner et al., Pharmacopsychiatry 21:301 (1988); Pomerleau et al., Addictive Behaviors 9:265 (1984); Onaivi et al., Life Sci. 54(3):193 (1994); Tripathi et al., J. Pharmacol. Exp. Ther. 221:91(1982) and Hamon, Trends in Pharmacol Res. 15:36 (1994).

Various nicotinic compounds have been reported as being useful for treating a wide variety of conditions and disorders. See, for example, Williams et al., Drug News Perspec. 7(4):205 (1994); Arneric et al., CNS Drug Rev. 1(1): 1 (1995); Arneric et al., Exp. Opin. Invest. Drugs 5(1):79 (1996); Bencherif et al., J. Pharmacol. Exp. Ther. 279:1413 (1996); Lippiello et al., J. Pharmacol. Exp. Ther. 279:1422 (1996); Damaj et al., Neuroscience (1997)J. Pharmacol. Exp. Ther. 291:390 (1999); Chiari et al., Anesthesiology 91:1447 (1999); Lavand'homme and Eisenbach, Anesthesiology 91:1455 (1999); Holladay et al., J. Med. ChemChem. 40(28): 4169 (1997); Bannon et al., Science 279: 77 (1998); PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al. Nicotinic compounds are reported as being particularly useful for treating a wide variety of CNS disorders. Indeed, a wide variety of compounds have been reported to have therapeutic properties. See, for example, U.S. Pat. Nos. 5,1871,166 to Kikuchi et al., 5,672,601 to Cignarella, PCT WO 99/21834 and PCT WO 97/40049, UK Patent Application GB 2295387 and European Patent Application 297,858.

CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses, and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a deficiency of choline, dopamine, norepinephrine and/or serotonin. Relatively common CNS disorders include pre-senile dementia (early-onset Alzheimer's disease), senile dementia (dementia of the Alzheimer's type), micro-infarct dementia, AIDS-related dementia, Creutzfeld-Jakob disease, Pick's disease, Parkinsonism including Parkinson's disease, progressive supranuclear palsy, Huntington's chorea, tardive dyskinesia, hyperkinesia, mania, attention deficit disorder, anxiety, dyslexia, schizophrenia, depression, obsessive-compulsive disorders and Tourette's syndrome.

It would be desirable to provide a useful method for the prevention and treatment of a condition or disorder by administering a nicotinic compound to a patient susceptible to or suffering from such a condition or disorder. It would be highly beneficial to provide individuals suffering from certain disorders (e.g., CNS diseases) with interruption of the symptoms of those disorders by the administration of a pharmaceutical composition containing an active ingredient having nicotinic pharmacology and which has a beneficial effect (e.g., upon the functioning of the CNS), but which does not provide any significant associated side effects. It would be highly desirable to provide a pharmaceutical composition incorporating a compound which interacts with nicotinic receptors, such as those which have the potential to effect the functioning of the CNS, but, when employed in an amount sufficient to effect the functioning of the CNS, does not significantly effect those receptor subtypes which have the potential to induce undesirable side effects (e.g., appreciable activity at cardiovascular and skeletal muscle sites).

### SUMMARY OF THE INVENTION

The present invention relates to aryl olefinic azacyclic compounds and aryl acetylenic azacylic compounds. The present invention relates in particular to five-membered heteroaromatic olefinic azacyclic compounds and five-membered heteroaromatic acetylenic azacylic compounds, including isoxazolyl olefinic cycloalkylamines and isoxazolyl acetylenic cycloalkylamines. The present invention also relates to prodrug derivatives of the compounds of the present invention.

Exemplary compounds of the present invention are 5-(2-(5-azabicyclo[3.3.0]octyl)vinyl)isoxazole and 5-(2-(5-azabicyclo[3.3.0]octyl)ethynyl)isoxazole. The compounds of the present invention function as agonists and bind specifically to certain nicotinic receptors. The present invention relates to methods for synthesizing these types of compounds.

The present invention also relates to methods for the prevention or treatment of a wide variety of conditions or disorders, and particularly those disorders characterized by dysfunction of nicotinic cholinergic neurotransmission including disorders involving neuromodulation of neurotransmitter release, such as dopamine release. The present invention also relates to methods for the prevention or treatment of disorders, such as central nervous system (CNS) disorders, which are characterized by an alteration in normal neurotransmitter release. The present invention also relates to methods for the treatment of certain conditions (e.g., a method for alleviating pain). The methods involve administering to a subject an effective amount of a compound of the present invention. As such, the present invention relates to a method for using the compounds of the present invention for the manufacture of pharmaceutical compositions for the treatment of a wide variety of diseases and disorders.

The present invention, in another aspect, relates to a pharmaceutical composition comprising an effective amount of a compound of the present invention. Such a pharmaceutical composition incorporates a compound which, when employed in effective amounts, has the capability of interacting with relevant nicotinic receptor sites of a subject, and hence has the capability of acting as a therapeutic agent in the prevention or treatment of a wide variety of conditions and disorders, particularly those disorders characterized by an alteration in normal neurotransmitter release. Preferred pharmaceutical compositions comprise compounds of the present invention.

The pharmaceutical compositions of the present invention are useful for the prevention and treatment of disorders, such as CNS disorders, which are characterized by an alteration of normal neurotransmitter release. The pharmaceutical compositions provide therapeutic benefit to individuals suffering from such disorders and exhibiting clinical manifestations of such disorders in that the compounds within those compositions, when employed in effective amounts, have the potential to: (i) exhibit nicotinic pharmacology and affect relevant nicotinic receptors sites (e.g., act as a pharmacological agonist to activate nicotinic receptors), and/or (ii) modulate neurotransmitter secretion and thus prevent and suppress the symptoms associated with those diseases. In addition, the compounds are expected to have the potential to fulfill the following results for the patient: (i) to alter the number of nicotinic cholinergic receptors of the brain of the patient, (ii) to exhibit neuroprotective effects and (iii) to result in no appreciable adverse side effects when administered in effective amounts—side effects such as significant increases in blood pressure and heart rate, significant negative effects upon the gastro-intestinal tract, and significant effects upon skeletal muscle. The pharmaceutical compositions of the present invention are believed to be safe and effective with regards to prevention and treatment of a wide variety of conditions and disorders.

The foregoing and other aspects of the present invention are explained in detail in the detailed description and examples set forth below.

### DETAILED DESCRIPTION OF THE INVENTION

The compounds of the present invention include compounds of the formula:

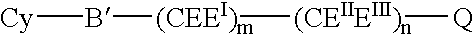

In the structure, Cy represents a 5-membered, preferably heteroaromatic ring, such as isoxazole, isothiazole, oxazole, thiazole, pyrazole, 1,2,4-oxadiazole and 1,2,4-triazole. Other examples of such rings are described in U.S. Pat. No. 6,022,868 to Olesen et al. the disclosure of which is incorporated herein by reference in its entirety. Cy is attached to B′ at any one of the various ring carbon atoms. The 5-membered heteroaromatic ring may bear one or more additional (i.e., in addition to B′) non-hydrogen substituent species.

One way of depicting Cy is as follows:

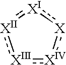

where X, X^{I}, X^{II}, X^{III} and X^{IV} are individually nitrogen, nitrogen bonded to oxygen, oxygen, sulfur or carbon bonded to a substituent species. Such non-hydrogen substituent species typically have a sigma m value of between −0.3 and 0.75 and include alkyl, substituted alkyl, alkenyl, substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo (e.g., F, Cl, Br, or I), —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6. R′ and R″ can together form a cycloalkyl functionality. Representative aromatic group-containing species include phenyl, benzyl, pyridinyl, pyrimidinyl, pyrazinyl, pyridazinyl, indolyl and quinolinyl. Other representative aromatic ring systems are set forth in Gibson et al., J. Med. Chem. 39:4065 (1996). When either R′ or R″ is a non-hydrogen substituent species, it may be further substituted, one or more times, by non-hydrogen substituent species, as described hereinbefore.

B′ is a substituted or unsubstituted two carbon bridging species; preferably can be acetylenic or ethylenic, and more preferably is ethylenic. That is, B′ can be selected from —CC— or —CR′═CR″—, wherein R′ and R″ are defined hereinbefore, but R′ and R″ preferably each are hydrogen. When the two carbon bridging species is ethylenic, that species can have a trans(E) or cis(Z) form, but most preferably is trans(E).

E, E′, E″ and E′″ individually represent hydrogen or a suitable non-hydrogen substituent (e.g., alkyl, substituted alkyl, halo-substituted alkyl, cycloalkyl, substituted cycloalkyl, heterocyclyl, substituted heterocyclyl, aryl, substituted aryl, alkylaryl, substituted alkylaryl, arylalkyl or substituted arylalkyl). E, E′, E″ and E′″ are preferably lower alkyl (e.g., straight chain or branched alkyl including C₁-C₈, preferably C₁-C₅, such as methyl, ethyl, or isopropyl) or halo substituted lower alkyl (e.g., straight chain or branched alkyl including C₁-C₈,preferably C₁-C₅, such as trifluoromethyl or trichloromethyl). Generally all of E, E′, E″ and E′″ are hydrogen, or at least one E, E′, E″ and E′″ is non-hydrogen and the remaining E, E′, E″ and E′″ are hydrogen. For example, when m is 1 and n is 0, E and E′ each can be hydrogen, or E can be hydrogen and E′ can be methyl; or when m is 1 and n is 1, E, E′, E″ and E′″ all can be hydrogen, or E, E′ and E″ can be hydrogen and E′″ can be methyl, or E′, E″ and E′″ can be hydrogen and E can be methyl. Typically, the selection of m, n, E, E′, E″ and E′″ is such that 0, 1 or 2, usually 0 or 1, and preferably 0, of the substituents designated as E, E′, E″ and E′″ are non-hydrogen (e.g., substituents such as alkyl or halo-substituted alkyl). However, it is preferred that when m is 1 and n is 0, neither E nor E′ are substituted or unsubstituted aryl, heteroaryl, benzhydryl or benzyl.

Q is one of the following azacycles:

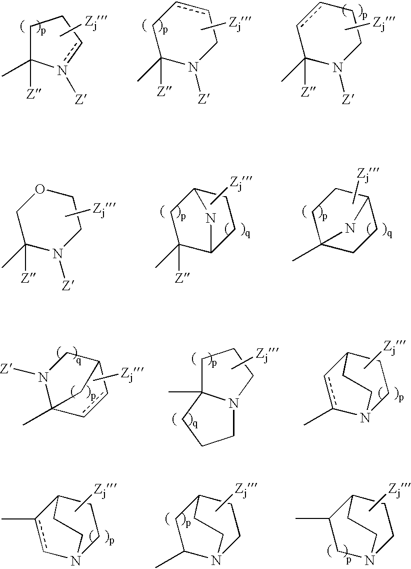

where Z′″ⱼ represents a suitable non-hydrogen substituent group (e.g., alkyl, substituted alkyl, halo-substituted alkyl, cycloalkyl, substituted cycloalkyl, heterocyclyl, substituted heterocyclyl, aryl, substituted aryl, alkylaryl, substituted alkylaryl, arylalkyl or substituted arylalkyl), but preferably alkyl. Z″ represents either hydrogen or lower alkyl. Z′ represents hydrogen, lower alkyl, acyl, alkoxycarbonyl, or aryloxycarbonyl. Preferably, Z′ is hydrogen or methyl and Z″ is hydrogen. In addition, j is an integer from 0 to 5, preferably 0 or 1, most preferably 0; p is 0, 1 or 2, preferably 0 or 1, and most preferably 1; and q is 0, 1, 2 or 3, preferably 0 or 1, and most preferably 1. The dotted line indicates that the bond between the two atoms can be either a single or a double bond. Suitable Q's are disclosed in U.S. Ser. No. 09/431,700 filed Nov. 1, 1999, the disclosure of which is incorporated herein by reference in its entirety.

As employed herein, “alkyl” refers to straight chain or branched alkyl radicals including C₁-C₅, preferably C₁-C₅, such as methyl, ethyl, or isopropyl; “substituted alkyl” refers to alkyl radicals further bearing one or more substituent groups such as hydroxy, alkoxy, mercapto, aryl, heterocyclo, halo, amino, carboxyl, carbamyl, cyano, and the like; “alkenyl” refers to straight chain or branched hydrocarbon radicals including C₁-C₈, preferably C₁-C₅ and having at least one carbon-carbon double bond; “substituted alkenyl” refers to alkenyl radicals further bearing one or more substituent groups as defined above; “cycloalkyl” refers to saturated or unsaturated cyclic ring-containing radicals containing three to eight carbon atoms, preferably three to six carbon atoms; “substituted cycloalkyl” refers to cycloalkyl radicals further bearing one or more substituent groups as defined above; “aryl” refers to aromatic radicals having six to ten carbon atoms; “substituted aryl” refers to aryl radicals further bearing one or more substituent groups as defined above; “alkylaryl” refers to alkyl-substituted aryl radicals; “substituted alkylaryl” refers to alkylaryl radicals further bearing one or more substituent groups as defined above; “arylalkyl” refers to aryl-substituted alkyl radicals; “substituted arylalkyl” refers to arylalkyl radicals further bearing one or more substituent groups as defined above; “heterocyclyl” refers to saturated or unsaturated cyclic radicals containing one or more heteroatoms (e.g., O, N, S) as part of the ring structure and having two to seven carbon atoms in the ring; and “substituted heterocyclyl” refers to heterocyclyl radicals further bearing one or more substituent groups as defined above.

Compounds of the present invention can occur as stereoisomeric structures, and the present invention relates to racemic mixtures of such compounds as well as single enantiomer compounds.

Representative compounds useful in carrying out the present invention include the following:

(E)-5-(2-(5-azabicyclo[3.3.0]octyl)vinyl)isoxazole

(Z)-5-(2-(5-azabicyclo[3.3.0]octyl)vinyl)isoxazole

5-(2-(5-azabicyclo[3.3.0]octyl)ethynyl)isoxazole

(E)-5-(2-(6-azabicyclo[4.3.0]nonyl)vinyl)isoxazole

(Z)-5-(2-(6-azabicyclo[4.3.0]nonyl)vinyl)isoxazole

5-(2-(6-azabicyclo[4.3.0]nonyl)ethynyl)isoxazole

(E)-5-(2-(6-azabicyclo[4.4.0]decyl)vinyl)isoxazole

(Z)-5-(2-(6-azabicyclo[4.4.0]decyl)vinyl)isoxazole

5-(2-(6-azabicyclo[4.4.0]decyl)ethynyl)isoxazole

(E)-5-(2-(6-azabicyclo[4.2.0]octyl)vinyl)isoxazole

(Z)-5-(2-(6-azabicyclo[4.2.0]octyl)vinyl)isoxazole

5-(2-(6-azabicyclo[4.2.0]octyl)ethynyl)isoxazole

(E)-5-(2-(5-azabicyclo[3.2.0]heptyl)vinyl)isoxazole

(Z)-5-(2-(5-azabicyclo[3.2.0]heptyl)vinyl)isoxazole

5-(2-(5-azabicyclo[3.2.0]heptyl)ethynyl)isoxazole

(E)-5-(2-(5-azabicyclo[3.3.0]octyl)vinyl)-1,2,4-oxadiazole

(Z)-5-(2-(5-azabicyclo[3.3.0]octyl)vinyl)-1,2,4-oxadiazole

(E)-5-(2-(5-azabicyclo[3.3.0]octyl)vinyl)-isothiazole

(Z)-5-(2-(5-azabicyclo[3.3.0]octyl)vinyl)-isothiazole

(E)-5-(2-(5-azabicyclo[3.3.0]octyl)vinyl)-1,3-oxazole

(Z)-5-(2-(5-azabicyclo[3.3.0]octyl)vinyl)-1,3-oxazole

(E)-5-(2-(5-azabicyclo[3.3.0]octyl)vinyl)pyrazole

(Z)-5-(2-(5-azabicyclo[3.3.0]octyl)vinyl)pyrazole

(E)-5-(2-(5-azabicyclo[3.3.0]octyl)vinyl)imidazole

(Z)-5-(2-(5-azabicyclo[3.3.0]octyl)vinyl)imidazole

(E)-5-(2-(5-azabicyclo[3.3.0]octyl)vinyl)1,2,5-oxadiazole

(Z)-5-(2-(5-azabicyclo[3.3.0]octyl)vinyl)1,2,5-oxadiazole

(E)-5-(2-(5-azabicyclo[3.3.0]octyl)vinyl)-1,2,3-triazole

(Z)-5-(2-(5-azabicyclo[3.3.0]octyl)vinyl)-1,2,4-triazole

(E)-5-(2-(5-azabicyclo[3.3.0]octyl)vinyl)thiazole

(Z)-5-(2-(5-azabicyclo[3.3.0]octyl)vinyl)thiazole

(E)-5-(2-pyrrolidin-2-ylvinyl)isoxazole

(Z)-5-(2-pyrrolidin-2-ylvinyl)isoxazole

5-(2-pyrrolidin-2-ylethynyl)isoxazole

(E)-5-(2-(2-piperidyl)vinyl)isoxazole

(Z)-5-(2-(2-piperidyl)vinyl)isoxazole

5-(2-(2-piperidyl)ethynyl)isoxazole

(E)-5-(1H,2H,3H,4H-2-azinyl)vinyl)isoxazole

(Z)-5-(1H,2H,3H,4H-2-azinyl)vinyl)isoxazole

5-(1H,2H,3H,4H-2-azinyl)ethynyl)isoxazole

(E)-5-(1H,2H,3H,6H-2-azinyl)vinyl)isoxazole

(Z)-5-(1H,2H,3H,6H-2-azinyl)vinyl)isoxazole

5-(1H,2H,3H,6H-2-azinyl)ethynyl)isoxazole

(E)-3-(2-isoxazol-5-ylvinyl)morpholine

(Z)-3-(2-isoxazol-5-ylvinyl)morpholine

3-(2-isoxazol-5-ylethynyl)morpholine

(E)-5-(2-(7-azabicyclo[2.2.1]hept-2-yl)vinyl)isoxazole

(Z)-5-(2-(7-azabicyclo[2.2.1]hept-2-yl)vinyl)isoxazole

5-(2-(7-azabicyclo[2.2.1]hept-2-yl)ethynyl)isoxazole

(E)-5-(2-(7-azabicyclo[2.2.1]heptyl)vinyl)isoxazole

(Z)-5-(2-(7-azabicyclo[2.2.1]heptyl)vinyl)isoxazole

5-(2-(7-azabicyclo[2.2.1]heptyl)ethynyl)isoxazole

(E)-5-(2-(2-azabicyclo[2.2.1]heptyl)vinyl)isoxazole

(Z)-5-(2-(2-azabicyclo[2.2.1]heptyl)vinyl)isoxazole

5-(2-(2-azabicyclo[2.2.1]heptyl)ethynyl)isoxazole

(E)-5-(2-quinuclidin-2-ylvinyl)isoxazole

(Z)-5-(2-quinuclidin-2-ylvinyl)isoxazole

5-(2-quinuclidin-2-ylethynyl)isoxazole

(E)-5-(2-quinuclidin-3-ylvinyl)isoxazole

(Z)-5-(2-quinuclidin-3-ylvinyl)isoxazole

5-(2-quinuclidin-3-ylethynyl)isoxazole

(E)-5-(2-(5-azabicyclo[3.2.2]non-2-yl)vinyl)isoxazole

(Z)-5-(2-(5-azabicyclo[3.2.2]non-2-yl)vinyl)isoxazole

5-(2-(5-azabicyclo[3.2.2]non-2-yl)ethynyl)isoxazole

(E)-5-(2-(5-azabicyclo[3.2.2]non-2-yl)vinyl)isoxazole

(Z)-5-(2-(5-azabicyclo[3.2.2]non-2-yl)vinyl)isoxazole

5-(1-(5-azabicyclo[3.2.2]non-2-yl)ethynyl)isoxazole

(E)-5-(2-(5-azabicyclo[3.2.2]non-6-yl)vinyl)isoxazole

(Z)-5-(2-(5-azabicyclo[3.2.2]non-6-yl)vinyl)isoxazole

5-(2-(5-azabicyclo[3.2.2]non-6-yl)ethynyl)isoxazole

(E)-5-(3-pyrrolidin-2-yl)prop-1-enyl)isoxazole

(Z)-5-(3-pyrrolidin-2-yl)prop-1-enyl)isoxazole

5-(3-pyrrolidin-2-yl)prop-1-enyl)isoxazole

(E)-5-(3-(2-piperidyl)prop-1-enyl)isoxazole

(Z)-5-(3-(2-piperidyl)prop-1-enyl)isoxazole

5-(3-(2-piperidyl)prop-1-enyl)isoxazole

(E)-3-isoxazol-5-ylprop-2-enyl)morpholine

(Z)-3-isoxazol-5-ylprop-2-enyl)morpholine

3-isoxazol-5-ylprop-2-enyl)morpholine

The manner in which compounds of the present invention are synthesized can vary. Compounds of the present invention can be prepared in either racemic form or in enantiomerically pure form. In one method, certain 5-membered heterocyclyl olefinic pyrrolidine compounds can be prepared by using a palladium-catalyzed coupling reaction of a 5-bromoisoxazole or 5-iodoisoxazole with an olefin possessing a protected pyrrolidine functionally, such as (2S)-2-allyl-1-tert-butoxycarbonylpyrrolidine (also known as (2S)-N-(tert-butoxycarbonyl)-2-(3-prop-1-enyl)pyrrolidine). Reaction conditions employing palladium(II) acetate, tri-o-tolylphosphine, and triethylamine, similar to those described by Frank et al., J. Org. Chem. 43(15): 2947 (1978) and Malek et al., J. Org. Chem. 47: 5395 (1982) can be used. The tert-butoxycarbonyl protecting group of the resulting reaction product, (2S)-(2E)-N-(tert-butoxycarbonyl)-2-(3-prop-1-(5-isoxazolyl)-1-enyl)pyrrolidine, can then be removed by treatment with a strong acid, such as trifluoroacetic acid, to produce (2S)-(2E)-5-(3-(pyrrolidin-2-yl)prop-1-enyl)isoxazole. The pyrrolidine ring can then be N-methylated using aqueous formaldehyde and sodium cyanoborohydride using methodology similar to that described by Abreo et al., J. Med. Chem. 39: 817 (1996) to afford (2S)-(2E)-5-(3-(1-methylpyrrolidin-2-yl)prop-1-enyl)isoxazole.

The aforementioned side chain, (2S)-2-allyl-N-(tert-butoxycarbonyl)pyrrolidine, can be prepared from commercially available (Aldrich Chemical Company) (2S)-2-pyrrolidinemethanol. The pyrrolidine nitrogen of the latter compound can be protected by treatment with di-tert-butyl dicarbonate in dichloromethane using triethylamine as a base to produce (2S)-N-(tert-butoxycarbonyl)-2-(hydroxymethyl)pyrrolidine. The latter compound can be treated with iodine, triphenylphosphine, and diethyl azodicarboxylate to give (2S)-N-(tert-butoxycarbonyl)-2-(iodomethyl)pyrrolidine. Treatment of the latter compound with vinylmagnesium bromide and copper (I) iodide produces the desired olefinic pyrrolidine, (2S)-2-allyl-N-tert-butoxycarbonylpyrrolidine.

Alternatively, treatment of (2S)-N-(tert-butoxycarbonyl)-2-(iodomethyl)pyrrolidine with lithium trimethylsilylacetylide, followed by deprotection using tetrabutylammonium fluoride, affords (2S)-N-(tert-butoxycarbonyl)-2-(propargyl)pyrrolidine. This can be coupled, using Sonogashira conditions (for example, see Yamanaka et al., Chem. Pharm. Bull. 29:3543 (1981)), with 5-bromoisoxazole or 5-iodoisoxazole, to give (2S)-(2E)-N-(tert-butoxycarbonyl)-5-(3-(pyrrolidin-2-yl)prop-1-ynyl)isoxazole. Typically tetrakis(triphenylphosphine)palladium(0) and copper(I) iodide are used in this coupling. Treatment with trifluoroacetic acid, as described above, will produce (2S)-(2E)-5-(3-(pyrrolidin-2-yl)prop-1-ynyl)isoxazole.

Using commercially available (2R)-2-pyrrolidinemethanol (Aldrich Chemical Company) and the reaction sequences described above, the corresponding R enantiomers of (2E)-5-(3-(pyrrolidin-2-yl)prop-1-enyl)isoxazole, (2E)-5-(3-(1-methylpyrrolidin-2-yl)prop-1-enyl)isoxazole and (2E)-5-(3-(pyrrolidin-2-yl)prop-1-ynyl)isoxazole can be made. Alternatively, enantiomerically pure 2-pyrrolidinemethanol can be synthetically elaborated to the aforementioned chiral olefinic pyrrolidine, 2-allyl-N-tert-butoxycarbonylpyrrolidine, using the methodology of Ikeda et al., Heterocycles 50: 31 (1999).

Certain compounds of the present invention, possessing an ethenyl or ethynyl linker between the heteroaromatic ring and the azacycle, can be prepared by a variety of methods. In one approach, using palladium-catalyzed coupling methods as described above, a 5-haloisoxazole such as a 5-bromoisoxazole is coupled with (2S)-N-(tert-butoxycarbonyl)-2-vinylpyrrolidine or (2S)-N-(tert-butoxycarbonyl)-2-ethynylpyrrolidine, and the tert-butoxycarbonyl protecting groups subsequently removed. The necessary precursors are readily prepared from (2S)-N-(tert-butoxycarbonyl)-2-(hydroxymethyl)pyrrolidine. Swern oxidation to produce the aldehyde (Swern et al., J. Org Chem. 41:3329 (1976)), followed by conversion to the olefin using the techniques described in Wittig et al., Liebigs Ann. 562:187 (1949), provides (2S)-N-(tert-butoxycarbonyl)-2-vinylpyrrolidine. Alternatively, (2S)-N-(tert-butoxycarbonyl)-2-ethynylpyrrolidine may be prepared by treatment of the aldehyde with carbon tetrabromide and triphenyl phosphine followed by n-butyllithium. The products of such a sequence, (2S)-(2E)-5-(2-pyrrolidin-2-ylvinyl)isoxazole and (2S)-5-(2-pyrrolidin-2-ylethynyl)isoxazole, can subsequently be methylated, as described previously, to produce the corresponding N-methyl derivatives. By using (2R)-2-pyrrolidinemethanol as the starting material for the sequence, the corresponding enantiomers of the above compounds can be prepared.

Alternatively, a Horner-Wadsworth-Emmons reaction between diethyl (5-isoxazolylmethyl)phosphonate and (2S)-N-(tert-butoxycarbonyl)-2-formylpyrrolidine which, upon deprotection, will provide (2S)-(2E)-3-(2-pyrrolidin-2-ylvinyl)isoxazole. For example, see Wadsworth et al., J. Am. Chem. Soc. 83:1733 (1961) and U.S. Pat. No. 6,022,868 to Olesen et al.

Other azacycles can easily be adapted to the aforementioned chemistry. For instance, the corresponding piperidinyl compound, (2E)-5-(2-piperidin-2-ylvinyl)isoxazole, can be prepared from commercially available 2-piperidinemethanol. Thus, sequential protection as the N-(tert-butoxycarbonyl) derivative, Swern oxidation, Wittig coupling and subsequent deprotection, completes the synthesis.

The manner in which 5-membered heteroaromatic olefinic azacyclic compounds, of the present invention, can be synthesized can vary. Those examples possessing a 1-azabicyclo[3.3.0]octane moiety can be synthesized utilizing 5-azabicyclo[3.3.0]octanecarboxaldehyde as a key intermediate in the synthetic pathway. Treatment of N-(tert-butoxycarbonyl)proline with sodium hydride, followed by methyl iodide, provides corresponding methyl ester in high yield. Sequential treatment with lithium diisopropylamine and 1,3-dibromopropane provides N-(tert-butoxycarbonyl)-2-(3-bromopropyl)-2-(methoxycarbonyl)pyrrolidine. Deprotection of the amine using hydrochloric acid, followed by intramolecular nucleophilic ring closure, provides methyl 5-azabicyclo[3.3.0]octanecarboxylate. Diisobutylaluminum hydride reduction of methyl 5-azabicyclo[3.3.0]octane carboxylate affords 5-azabicyclo[3.3.0]octanecarboxaldehyde. A Homer-Wadsworth-Emmons reaction between diethyl (5-isoxazolylmethyl)phosphonate and 5-azabicyclo[3.3.0]octane carboxaldehyde provides (E)-5-(2-(5-azabicyclo[3.3.0]octyl)vinyl)isoxazole. Diethyl (5-isoxazolylmethyl)phosphonate is prepared according to the method described in Deshong et al., J. Org. Chem. 53: 1356 (1988). Alternatively, the treatment of 5-azabicyclo[3.3.0]octanecarboxaldehyde with 5-(lithiomethyl)isoxazole and dehydration of the resulting alcohol, as described in U.S. Pat. No. 6,022,868 to Olesen et al., will provide (E)-5-(2-(5-azabicyclo[3.3.0]octyl)vinyl)isoxazole.

Compounds of the present invention include those in which the isoxazole ring is substituted (e.g., on the 3and 4 position) with moieties that are stable to the processes used in their generation. For instance, treatment of 5-azabicyclo[3.3.0]octanecarboxaldehyde with the anion of 5-(diethylphosphonylmethyl)-3-methylisoxazole will provide (E)-5-(2-(5-azabicyclo[3.3.0]octyl)vinyl)-3-methylisoxazole in a similar manner to that described hereinbefore. 5-Diethylphosphonylmethyl-3-methylisoxazole can be prepared as described in Lee et al., Syn. Commun. 29: 3621 (1999) and Lee et al., Synthesis 2027 (1999). Alternatively, treatment of 3-methyl-5-(trimethylsilyl(lithiomethyl))isoxazole with 5-azabicyclo[3.3.0]octanecarboxaldehyde will provide (E)-5-(2-(5-azabicyclo[3.3.0]octyl)vinyl)isoxazole. Techniques such as those described in U.S. Pat. No. 6,022,868 to Olesen et al. can be used. Heteroaromtic olefinic azabicyclic compounds containing other five-membered heterocycles can be prepared using Horner-Wadsworth-Emmons reaction chemistry as described in U.S. Pat. No. 6,022,868 to Olesen et al. Alternatively, condensation of 5-azabicyclo[3.3.0]octanecarboxaldehyde with a 5-membered (heterocyclyl)methyllithium followed by dehydration of the resulting alcohol will also provide the desired compounds. Representative examples of 5-membered (heterocyclyl)methyllithium species are described by Micetich et al., Can. J. Chem. 48: 2006 (1970). Other five-membered heteroaromatic ethenyl azabicyclic compounds can be synthesized from the trimethylsilylmethyl derivatives of 5-membered ring heterocycles. Thus, condensation of chlorotrimethylsilane with 5-membered (heterocyclyl)methyllithiums gives trimethylsilylmethyl-substituted heterocycles, which can be deprotonated with n-butyllithium. For example see Nesi et al., J. Organomet. Chem. 195: 275 (1980). Treatment of the these carbanions with 5-azabicyclo[3.3.0]octanecarboxaldehyde will lead to the desired compounds of the present invention.

The azabicycle moiety in the present invention can vary as well. For example, (E)-5-(2-(6-azabicyclo[4.3.0]nonyl)vinyl)isoxazole and (E)-5-(2-(5-azabicyclo[3.2.0]heptyl)vinyl)isoxazole can be prepared in a similar manner as described. Treatment of N-(tert-butoxycarbonyl)-2-(methoxycarbonyl)pyrrolidine with lithium diisopropylamine, followed by reaction with 1,4-dibromobutane, can provide N-(tert-butoxycarbonyl)-2-(4-bromobutyl)-2-(methoxycarbonyl)pyrrolidine which, upon deprotection of the amine, followed by intramolecular nucleophilic substitution and diisobutylaluminum hydride reduction of the ester can provide 6-azabicyclo[4.3.0]nonanecarboxaldehyde. A Horner-Wadsworth-Emmons reaction with the anion of 5-diethylphosphonylmethyl-3-methylisoxazole will provide (E)-5-(2-(6-azabicyclo[4.3.0]nonyl)vinyl)-3-methylisoxazole. By substituting 1,4-dibromobutane in the above scheme with 1,2-dibromoethane (or synthetic equivalent), (E)-5-(2-(5-azabicyclo[3.2.0]heptyl)vinyl)isoxazole can be prepared.

Other azabicyclic compounds can be prepared from pipecolinic acid, in a manner similar to that just described. Thus, conversion to the methyl ester and protection of the amine as its N-(tert-butoxycarbonyl) derivative gives a material, N-(tert-butoxycarbonyl)-2-(methoxycarbonyl)piperidine, which is homologous to the previously described N-(tert-butoxycarbonyl)-2-(methoxycarbonyl)pyrrolidine. Subsequent employment of the annulation sequence of anion formation, alkylation with a 1,4-dibromobutane, and deprotection of the nitrogen (with concommitent ring closure), will provide methyl 6-azabicyclo[4.4.0]decanecarboxylate. Reduction to the aldehyde (with diisobutylaluminum hydride, and subsequent condensation with the anion of diethyl (5-isoxazolylmethyl)phosphonate, will give (E)-5-(2-(6-azabicyclo[4.4.0]decyl)vinyl)isoxazole. Other azabicyclic ring examples of the present invention can be made in a similar fashion, by utilizing different amino acid and dihalide starting materials.

The methods by which 5-membered heteroaromatic alkynyl azacyclic compounds are produced can vary. For example, treatment of 5-azabicyclo[3.3.0]octanecarboxaldehyde with carbon tetrabromide in carbon tetrachloride solution will provide 1-aza-5-(2,2-dibromovinyl)bicyclo[3.3.0]octane which, upon treatment with n-butyllithium, will provide 1-aza-5-ethynylbicyclo[3.3.0]octane. Alternatively, treatment of 5-azabicyclo[3.3.0]octanecarboxaldehyde and (dichloromethyl)diethoxyphosphino-1-one with lithium diisopropylamine, followed by n-butyllithium, will provide 1-aza-5-ethynylbicyclo[3.3.0]octane. Sonogashira coupling of a 5-bromoisoxazole with 1-aza-5-ethynylbicyclo[3.3.0]octane will provide 5-(2-(5-azabicyclo[3.3.0]octyl)ethynyl)isoxazole. For example, see Yamanaka et al., Chem. Pharm. Bull. 29:3543 (1981). Other azabicyclocarboxaldes described herein can be utilized similarly.

There are a number of methods by which the (Z)-olefinic isomers of 5-membered heteroaromatic olefinic azacyclic compounds can be synthetically produced. In one approach, these (Z)-olefinic isomers can be prepared by the controlled hydrogenation of the corresponding alkynyl compounds using commercially available Lindlar catalyst (Aldrich Chemical Company) according to the methodology set forth in Lindlar et al., Org. Syn. 46: 89 (1966). In some cases, the heterocyclic group may be reactive under these hydrogenation conditions, leading to undesired products. In these instances, mixtures of the (E) and (Z) olefinic compounds can be prepared by reaction of the aldyhydes with Wittig reagents. Subsequent chromatographic separation will provide samples of both geometric isomers. For examples of the preparation of olefins by the Wittig reaction, see House et al., J. Org. Chem 29:3327 (1964).

In a similar manner, 2-allylquinuclidine can be subjected to a palladium-catalyzed coupling reaction with a 5-haloisoxazole, such as 5-bromoisoxazole, to afford 2-(1-(5-isoxazolyl)propen-3-yl)quinuclidine. The precursor 2-allylquinuclidine can be prepared from 3-quinuclidinone (commercially available from Aldrich Chemical Company) by alkylation and modified Wolff-Kishner reduction, as described in Forsyth et al., J. Am. Chem. Soc. 109:7270 (1987). Thus, 3-quinuclidinone can be converted to the corresponding imine with isopropylamine and molecular sieves. Alkylation of the imine with lithium diisopropylamine and allyl bromide, followed by hydrolysis, produces 2-allyl-3-quinuclidinone. Removal of the carbonyl-protecting group can then be effected by converting the ketone into the p-toluenesulfonylhydrazone, followed by reduction with sodium cyanoborohydride, to afford 2-allylquinuclidine.

The manner in which certain 5-membered heterocyclic-substituted olefinic amine compounds possessing an azetidinyl moiety are synthesized can vary. Using one synthetic approach, 5-(2-(2-azetidinyl)vinyl)isoxazole can be synthesized starting from commercially azetidine-2-carboxylic acid (Aldrich Chemical Company). Azetidine-2-carboxylic acid can be reduced by any of a number of methods common to the art, such as treatment with lithium aluminum hydride, to give azetidine-2-methanol. Protection of the azetidinyl nitrogen of the latter compound can be accomplished by treatment with tert-butylpyrocarbonate and base to give N-(tert-butyloxycarbonyl)-2-(hydroxymethyl)azetidine, using methodology similar to that described by Carpino et al., Acc. Chem. Res, 6:191 (1973). This alcohol can be oxidized by a Swern oxidation to the corresponding aldehyde. A Homer-Wadsworth-Emmons reaction between the aldehyde and diethyl (5-isoxazolylmethyl)phosphonate, followed by deprotection, will provide compounds of the present invention.

The manner in which certain 5-membered heteroaromatic olefinic amine compounds, possessing an azabicyclo[2.2.1]heptane functionality, are synthesized can vary. 2-(2-(3-Isoxazolyl)vinyl-7-azabicyclo[2.2.1]heptane can be synthesized starting with ethyl 7-aza-7-(ethoxycarbonyl)bicyclo[2.2.1]heptane-2-carboxylate, which can be generated from commercially avaliable tropinone (Lancaster Chemical Company) according to the method of Badio et al., Eur. J. Pharmacol., 321:865 (1997). This compound can then be reduced to ethyl 7-aza-2-(formyl)bicyclo[2.2.1]heptane-7-carboxylate using one equivalent of diisobutylaluminum hydride. A Horner-Wadsworth-Emmons reaction between the aldehyde and diethyl (5-isoxazolylmethyl)phosphonate, followed by deprotection, will provide 2-(2-(5-isoxazolyl)vinyl-7-azabicyclo[2.2.1]heptane.

The manner in which certain aryl-substituted olefinic amine compounds possessing a 2-azabicyclo[2.2.1]heptane moiety are synthesized can vary. In one synthetic approach, ethyl 3-aza-3-((4-toluenesulfonyl)bicyclo[2.2.1]hept-5-ene-2-carboxylate, synthesized according to the method of Hamley et al., Synlett. 29 (1991), can be reduced to 2-aza-3-(hydroxymethyl)-2-((4-toluenesulfonyl)bicyclo[2.2.1]hept-5-ene using an excess of diisobutyllithium hydride at 0° C. Reduction of the olefin can be accomplished by various methods known to those skilled in the art, such as hydrogenation over palladium catalyst, to give 2-aza-3-(hydroxymethyl)-2-((4-toluenesulfonyl)bicyclo[2.2.1]heptane. This alcohol can then be oxidized to 2-aza-3-(formyl)-2-((4-toluenesulfonyl)bicyclo[2.2.1]heptane using oxalyl chloride and dimethylsulfoxide. A Horner-Wadsworth-Emmons reaction between the aldehyde and diethyl (5-isoxazolylmethyl)phosphonate, followed by deprotection by treatment of the aforementioned N-tosylate with sodium naphthylide according to the procedure of Ji et al., J. Am. Chem. Soc. 89:5311 (1967), will provide compounds of the present invention.

The present invention relates to a method for providing prevention of a condition or disorder to a subject susceptible to such a condition or disorder, and for providing treatment to a subject suffering therefrom. For example, the method comprises administering to a patient an amount of a compound effective for providing some degree of prevention of the progression of a CNS disorder (i.e., provide protective effects), amelioration of the symptoms of a CNS disorder, and amelioration of the recurrence of a CNS disorder. The method involves administering an effective amount of a compound selected from the general formulae, which are set forth hereinbefore. The present invention relates to a pharmaceutical composition incorporating a compound selected from the general formulae, which are set forth hereinbefore. Optically active compounds can be employed as racemic mixtures or as pure enantiomers. The compounds can be employed in a free base form or in a salt form (e.g., as pharmaceutically acceptable salts). Examples of suitable pharmaceutically acceptable salts include inorganic acid addition salts such as hydrochloride, hydrobromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with an acidic amino acid such as aspartate and glutamate; alkali metal salts such as sodium and potassium; alkaline earth metal salts such as magnesium and calcium; ammonium salt; organic basic salts such as trimethylamine, triethylamine, pyridine, picoline, dicyclohexylamine, and N,N′-dibenzylethylenediamine; and salts with a basic amino acid such as lysine and arginine. The salts may be in some cases hydrates or ethanol solvates. Representative salts are provided as described in U.S. Pat. Nos. 5,597,919 to Dull et al., 5,616,716 to Dull et al. and 5,663,356 to Ruecroft et al., the disclosures of which are incorporated herein by reference in their entirety.

Compounds of the present invention are useful for treating those types of conditions and disorders for which other types of nicotinic compounds have been proposed as therapeutics. See, for example, Williams et al., Drug News Perspec. 7(4):205 (1994), Arneric et al., CNS Drug Rev. 1(1):1 (1995), Arneric et al., Exp. Opin. Invest. Drugs 5(1):79 (1996), Bencherif et al., J. Pharmacol. Exp. Ther. 279:1413 (1996), Lippiello et al., J. Pharmacol. Exp. Ther. 279:1422 (1996), Damaj et al., J. Pharmacol. Exp. Ther. 291:390 (1999); Chiari et al., Anesthesiology 91:1447 (1999); Lavand'homme and Eisenbach, Anesthesiology 91:1455 (1999); Neuroscience (1997), Holladay et al., J. Med. Chem. 40(28):4169 (1997), Bannon et al., Science 279:77 (1998), PCT WO 94/08992, PCT WO 96/31475, and U.S. Pat. Nos. 5,583,140 to Bencherif et al., 5,597,919 to Dull et al., and 5,604,231 to Smith et al., the disclosures of which are incorporated herein by reference in their entirety. Compounds of the present invention can be used as analgesics, to treat ulcerative colitis, inflammatory and auto-immune diseases (e.g., arthritis, cholangitis, stomatitis, pouchitis, viral pneumonitis), to treat a variety of neurodegenerative diseases, and to treat convulsions such as those that are symptomatic of epilepsy. CNS disorders which can be treated in accordance with the present invention include pre-senile dementia (early onset Alzheimer's disease), senile dementia (dementia of the Alzheimer's type), HIV-dementia, multiple cerebral infarcts, Parkinsonism including Parkinson's disease, Pick's disease, Huntington's chorea, tardive dyskinesia, hyperkinesia, mania, attention deficit disorder, anxiety, depression, mild cognitive impairment, dyslexia, schizophrenia and Tourette's syndrome. Compounds of the present invention also can be used to treat conditions such as syphillis and Creutzfeld-Jakob disease. The compounds of the present invention also can be appropriately synthesized and used as or within pharmaceutical compositions that are used as diagnostic probes.

The pharmaceutical composition also can include various other components as additives or adjuncts. Exemplary pharmaceutically acceptable components or adjuncts which are employed in relevant circumstances include antioxidants, free-radical scavenging agents, peptides, growth factors, antibiotics, bacteriostatic agents, immunosuppressives, anticoagulants, buffering agents, anti-inflammatory agents, anti-pyretics, time-release binders, anaesthetics, steroids, vitamins, minerals and corticosteroids. Such components can provide additional therapeutic benefit, act to affect the therapeutic action of the pharmaceutical composition, or act towards preventing any potential side effects, which may be imposed as a result of administration of the pharmaceutical composition. In certain circumstances, a compound of the present invention can be employed as part of a pharmaceutical composition with other compounds intended to prevent or treat a particular disorder.

The manner in which the compounds are administered can vary. The compounds can be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); orally (e.g., in liquid form within a solvent such as an aqueous or non-aqueous liquid, or within a solid carrier); intravenously (e.g., within a dextrose or saline solution); as an infusion or injection (e.g., as a suspension or as an emulsion in a pharmaceutically acceptable liquid or mixture of liquids); intrathecally; intracerebroventricularly; or transdermally (e.g., using a transdermal patch). Although it is possible to administer the compounds in the form of a bulk active chemical, it is preferred to present each compound in the form of a pharmaceutical composition or formulation for efficient and effective administration. Exemplary methods for administering such compounds will be apparent to the skilled artisan. For example, the compounds can be administered in the form of a tablet, a hard gelatin capsule or as a time-release capsule. As another example, the compounds can be delivered transdermally using the types of patch technologies available from Novartis and Alza Corporation. The administration of the pharmaceutical compositions of the present invention can be intermittent or at a gradual, continuous, constant or controlled rate to a warm-blooded animal (e.g., a mammal such as a mouse, rat, cat, rabbit, dog, pig, cow, or monkey), but advantageously is administered preferably to a human being. In addition, the time of day and the number of times per day that the pharmaceutical formulation is administered can vary. Preferable administration is such that the active ingredients of the pharmaceutical formulation interact with receptor sites within the body of the subject that affect the functioning of the CNS. More specifically, in treating a CNS disorder, preferable administration is designed to optimize the effect upon those relevant receptor subtypes that have an effect upon the functioning of the CNS, while minimizing the effects upon muscle-type receptor subtypes. Other suitable methods for administering the compounds of the present invention are described in U.S. Pat. No. 5,604,231 to Smith et al.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. Thus, when treating a CNS disorder, an effective amount of compound is an amount sufficient to pass across the blood-brain barrier of the subject, to bind to relevant receptor sites in the brain of the subject and to activate relevant nicotinic receptor subtypes (e.g., provide neurotransmitter secretion, thus resulting in effective prevention or treatment of the disorder). Prevention of the disorder is manifested by delaying the onset of the symptoms of the disorder. Treatment of the disorder is manifested by a decrease in the symptoms associated with the disorder or an amelioration of the recurrence of the symptoms of the disorder.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to activate relevant receptors to effect neurotransmitter (e.g., dopamine) release, but the amount should be insufficient to induce effects on skeletal muscles and ganglia to any significant degree. The effective dose of compounds will of course differ from patient to patient, but in general includes amounts starting where CNS effects or other desired therapeutic effects occur but below the amount where muscular effects are observed.

The compounds useful according to the method of the present invention have the ability to pass across the blood-brain barrier of the patient. As such, such these compounds have the ability to enter the central nervous system of the patient. The log P values of typical compounds, which are useful in carrying out the present invention, are generally greater than about −0.5, often are greater than about 0, and frequently are greater than about 0.5. The log P values of such typical compounds generally are less than about 3, often are less than about 2, and frequently are less than about 1. Log P values provide a measure of the ability of a compound to pass across a diffusion barrier, such as a biological membrane, including the blood brain barrier. See, for example, Hansch et al., J. Med. Chem. 11:1 (1968).

The compounds useful according to the method of the present invention have the ability to bind to, and in most circumstances, cause activation of, nicotinic dopaminergic receptors of the brain of the patient. As such, these compounds have the ability to express nicotinic pharmacology and, in particular, to act as nicotinic agonists. The receptor binding constants of typical compounds useful in carrying out the present invention generally exceed about 0.1 nM, often exceed about 1 nM, and frequently exceed about 10 nM. The receptor binding constants of certain compounds are less than about 100 μM, often are less than about 10 μM and frequently are less than about 5 μM; and of preferred compounds generally are less than about 2.5 μM, sometimes are less than about 1 μM, and can be less than about 100 nM. Receptor binding constants provide a measure of the ability of the compound to bind to half of the relevant receptor sites of certain brain cells of the patient. See, for example, Cheng et al., Biochem. Pharmacol. 22:3099 (1973).

The compounds useful according to the method of the present invention have the ability to demonstrate a nicotinic function by effectively activating neurotransmitter secretion from nerve ending preparations (i.e., synaptosomes). As such, these compounds have the ability to activate relevant neurons to release or secrete acetylcholine, dopamine, and other neurotransmitters. Generally, typical compounds useful in carrying out the present invention provide for the activation of dopamine secretion in amounts of at least one third, typically at least about 10 times less, frequently at least about 100 times less, and sometimes at least about 1,000 times less than those required for activation of muscle-type nicotinic receptors. Certain compounds of the present invention can provide secretion of dopamine in an amount which is comparable to that elicited by an equal molar amount of (S)-(−)-nicotine.

The compounds of the present invention, when employed in effective amounts in accordance with the method of the present invention, are selective to certain relevant nicotinic receptors, but do not cause significant activation of receptors associated with undesirable side effects at concentrations at least greater than those required for activation of dopamine release. By this is meant that a particular dose of compound resulting in prevention and/or treatment of a CNS disorder is essentially ineffective in eliciting activation of certain ganglionic-type nicotinic receptors at concentration higher than 5 times, preferably higher than 100 times, and more preferably higher than 1,000 times than those required for activation of dopamine release. This selectivity of certain compounds of the present invention against those ganglionic-type receptors responsible for cardiovascular side effects is demonstrated by a lack of the ability of those compounds to activate nicotinic function of adrenal chromaffin tissue at concentrations greater than those required for activation of dopamine release.

Compounds of the present invention, when employed in effective amounts in accordance with the method of the present invention, are effective towards providing some degree of prevention of the progression of CNS disorders, amelioration of the symptoms of CNS disorders, and amelioration to some degree of the recurrence of CNS disorders. However, such effective amounts of those compounds are not sufficient to elicit any appreciable side effects, as demonstrated by increased effects relating to skeletal muscle. As such, administration of certain compounds of the present invention provides a therapeutic window in which treatment of certain CNS disorders is provided and certain side effects are avoided. That is, an effective dose of a compound of the present invention is sufficient to provide the desired effects upon the CNS but is insufficient (i.e., is not at a high enough level) to provide undesirable side effects. Preferably, effective administration of a compound of the present invention resulting in treatment of CNS disorders occurs upon administration of less than ⅕, and often less than 1/10, that amount sufficient to cause certain side effects to any significant degree.

The pharmaceutical compositions of the present invention can be employed to prevent or treat certain other conditions, diseases and disorders. Exemplary of such diseases and disorders include inflammatory bowel disease, pouchitis, acute cholangitis, aphthous stomatitis, arthritis (e.g., rheumatoid arthritis and osteoarthritis), neurodegenerative diseases, cachexia secondary to infection (e.g., as occurs in AIDS, AIDS-related complex and neoplasia), as well as those indications set forth in PCT WO 98/25619. The pharmaceutical compositions of the present invention can be employed in order to ameliorate many of the symptoms associated with those conditions, diseases and disorders. Thus, pharmaceutical compositions of the present invention can be used in treating genetic diseases and disorders, in treating auto-immune disorders such as lupus, as anti-infectious agents (e.g., for treating bacterial, fungal and viral infections, as well as the effects, such as sepsis, of other types of toxins), as anti-inflammatory agents (e.g., for treating acute cholangitis, aphthous stomatitis, asthma, and ulcerative colitis), and as inhibitors of cytokine release (e.g., as is desirable in the treatment of cachexia, inflammation, neurodegenerative diseases, viral infection, and neoplasia). The compounds of the present invention can also be used as adjunct therapy in combination with existing therapies in the management of the aforementioned types of diseases and disorders. In such situations, preferable administration is such that the active ingredients of the pharmaceutical formulation act to optimize effects upon abnormal cytokine production, while minimizing effects upon receptor subtypes such as those that are associated with muscle and ganglia. Preferable administration is such that active ingredients interact with regions where cytokine production is affected or occurs. For the treatment of such conditions or disorders, compounds of the present invention are very potent (i.e., affect cytokine production and/or secretion at very low concentrations) and are very efficacious (i.e., significantly inhibit cytokine production and/or secretion to a relatively high degree).

Most preferably, effective doses are at very low concentrations, where maximal effects are observed to occur. Concentrations, determined as the amount of compound per volume of relevant tissue, typically provide a measure of the degree to which that compound affects cytokine production. Typically, the effective dose of compounds generally requires administering the compound in an amount of less than 5 mg/kg of patient weight. Often, the compounds of the present invention are administered in an amount from less than about 1 mg/kg patent weight and usually less than about 100 μg/kg of patient weight, but frequently between about 10 μg to less than 100 μg/kg of patient weight. For compounds of the present invention that do not induce effects on muscle-type nicotinic receptors at low concentrations, the effective dose is less than 5 mg/kg of patient weight; and often such compounds are administered in an amount from 50 μg to less than 5 mg/kg of patient weight. The foregoing effective doses typically represent that amount administered as a single dose, or as one or more doses administered over a 24-hour period.

For human patients, the effective dose of typical compounds generally requires administering the compound in an amount of at least about 1, often at least about 10, and frequently at least about 25 μg/24 hr/patient. For human patients, the effective dose of typical compounds requires administering the compound which generally does not exceed about 500, often does not exceed about 400, and frequently does not exceed about 300 μg/24 hr/patient. In addition, administration of the effective dose is such that the concentration of the compound within the plasma of the patient normally does not exceed , 500 pg/mL, often does not exceed 300 pg/mL, and frequently does not exceed 100 pg/mL. When employed in such a manner, compounds of the present invention are dose dependent, and, as such, cause inhibition of cytokine production and/or secretion when employed at low concentrations but do not exhibit those inhibiting effects at higher concentrations. Compounds of the present invention exhibit inhibitory effects upon cytokine production and/or secretion when employed in amounts less than those amounts necessary to elicit activation of relevant nicotinic receptor subtypes to any significant degree.

The following examples are provided to illustrate the present invention and should not be construed as limiting the scope thereof. In these examples, all parts and percentages are by weight, unless otherwise noted. Reaction yields are reported in mole percentages.

### EXAMPLES

### Assays

Determination of Binding to Relevant Receptor Sites

Binding of the compounds to relevant receptor sites was determined in accordance with the techniques described in U.S. Pat. No. 5,597,919 to Dull et al. Inhibition constants (Kᵢ values), reported in nM, were calculated from the IC₅₀ values using the method of Cheng et al., Biochem. Pharmacol. 22:3099 (1973). Low binding constants indicate that the compounds of the present invention exhibit good high affinity binding to certain CNS nicotinic receptors. All final compounds of the present invention reported herein have acceptable binding to the relevant receptor sites.

### Example 1

Sample No. 1 is (E)-5-(2-(5-azabicyclo[3.3.0]octyl)vinyl)isoxazole, which was prepared in accordance with the following techniques:

5-(Bromomethyl)isoxazole

N-bromosuccinimide (21.4 g, 120 mmole), 5-methylisoxazole (9.97 g, 120 mmole) and benzoylperoxide (2.41 g, 12.0 mmole) in carbon tetrachloride (250 mL) were heated while stirring at 80° C. for 6 h. The reaction mixture was filtered and then concentrated by rotary evarporation. Distillation at reduced pressure (bp 54-57° C., 0.5 mm Hg) provided pure product as a colorless oil (14.00 g, 72% yield).

Diethyl 5-(isoxazolylmethyl)phosphonate

5-(Bromomethyl)isoxazole (5.38 g, 33.2 mmole) was stirred at 0° C. as triethylphosphite (5.7 mL, 33.2 mmole) was slowly added. The mixture was stirred at room temperature for 48 h, heated under reflux for 24 h, and then concentrated by rotary evaporation. Purification by distillation at reduced pressure (bp 109-115° C., 0.04 mm Hg) provided pure product as a colorless oil (6.75 g, 92.7% yield).

Methyl (2S)-N-(tert-butoxycarbonyl)pyrrolidine-2-carboxylate

A solution of N-(tert-butoxycarbonyl)-(L)-proline (10.00 g, 46.44 mmole) and iodomethane (16.48 g, 116.1 mmole) in dimethylformamide (150 mL) was stirred at 0° C. as an 80% dispersion of sodium hydride in mineral oil (3.48 g, 116 mmole) was added in portions over 45 min. The mixture was allowed to stir at 25° C. for 24 h, then poured onto water (50 mL) and extracted with methylene chloride (3×75 mL). The combined extracts were dried (Na₂SO₄) and concentrated by rotary evaporation. Chromatography on silica gel, using ethyl acetate:hexane (1:10) as eluent, provided pure material as a viscous yellow oil (9.23 g, 86.7%)

Methyl (2S)-2-(3-bromopropyl)-N-(tert-butoxycarbonyl)pyrrolidine-2-carboxylate

A 2.5 M solution of n-butyllithium in hexanes (20.1 mL, 24.0 mmole) was added to a stirred solution of diisopropylamine (4.11 g, 24.0 mmole) in dry tetrahydrofuran (20 mL) at 0° C. The mixture was stirred for 15 min, then cooled to −78° C. and methyl (2S)-N-(tert-butoxycarbonyl)pyrrolidine-2-carboxylate was added via syringe. The mixture was stirred at −78° C. for 1 h, and then 1,3-dibromopropane (6.50 g, 24.0 mmol) was added via syringe and the mixture was stirred at −78° C. for 2 h. The mixture warmed to 25° C. and stirred for 24 h. It was then poured into saturated aqueous NH₄Cl solution (50 mL) and extracted with methylene chloride (5×35 mL). The combined extracts were dried (Na₂SO₄) and concentrated by rotary evaporation. Chromatography on silica gel, using a gradient of ethyl acetate:hexane (1:10 to 2:10) as eluent, provided pure material as a viscous yellow oil (5.84 g, 76.3

Methyl 5-azabicyclo[3.3.0]octanecarboxylate

A solution of methyl (2S)-2-(3-bromopropyl)-N-(tert-butoxycarbonyl)pyrrolidine-2-carboxylate (2.93 g, 0.840 mmole) in ethyl acetate (50 mL) was treated with concentrated hydrochloric acid (15 mL) and allowed to stir at 25° C. for 1.5 h. The mixture was poured onto water (50 mL) and the layers separated. The aqueous portion was carefully adjusted to pH 12 using solid K₂CO₃ and allowed to stir at 25° C. for 1 h. The mixture was extracted with methylene chloride (4×25 mL). The combined extracts were dried (Na₂SO₄) and concentrated by rotary evaporation to afford 0.566 g (39.8% yield) of a yellow oil.

5-Azabicyclo[3.3.0]octanecarboxaldehyde

A solution of methyl 5-azabicyclo[3.3.0]octanecarboxylate (1.35 g, 7.99 mmole) in toluene (35 mL) at −78° C. was treated with a 1.5 M solution of diisobutylaluminum hydride in toluene (6.37 mL, 4.01 mmole), and the mixture was allowed to stir at −78° C. for 6 h. The mixture was quenched with 10% aqueous hydrochloric acid solution (10 mL) and then extracted with methylene chloride (35 mL). The aqueous portion was carefully adjusted to pH 12 using solid Na₂CO₃ and then extracted with methylene chloride (5×25 mL). The combined extracts were dried (Na₂SO₄) and concentrated by rotary evaporation. Chromatography on silica gel, using acetone:chloroform (1:5) as eluent, provided pure product (0.566 g, 39.8% yield) as a yellow oil.

(E)-5-(2-(5-Azabicyclo[3.3.0]octyl)vinyl)isoxazole

A solution of diethyl 5-(isoxazolylmethyl)phosphonate (0.197 g, 0.90 mmole) in dry tetrahydrofuran (10 mL) at 0° C. was treated with a 2.5M solution of n-butyllithium in hexanes (0.36 mL, 0.90 mmole) and stirred for 0.5 h. 5-Azabicyclo[3.3.0]octanecarboxaldehyde (0.0837 g, 0.60 mmole) was added and the mixture was allowed to stir at 25° C. for 4 h, then poured onto water (10 mL) and extracted with methylene chloride (3×25 mL). The combined extracts were dried (Na₂SO₄) and concentrated by rotary evaporation. Chromatography on silica gel, using methanol:chloroform (1:10) as eluent, provided slightly impure product as an oil. A chloroform solution of the material was treated with 10% aqueous hydrochloric acid (3×15 mL). The combined aqueous portion was carefully adjusted to pH 12 using solid K₂CO₃ and then extracted with methylene chloride (5×25 mL). The combined extracts were dried (Na₂SO₄) and concentrated by rotary evaporation to provide pure product (0.0578 g, 47.2% yield) as a yellow oil.

Sample No. 1 exhibits a Kᵢ of 140 nM. The low binding constant indicates that the compound exhibits good high-affinity binding to certain CNS nicotinic receptors.

The foregoing is illustrative of the present invention and is not to be construed as limiting thereof. The invention is defined by the following claims, with equivalents of the claims to be included therein.

That which is claimed is:

1. A compound of the formula: 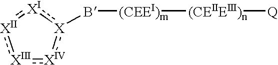
where each of X, X^{I}, X^{II}, X^{III} and X^{IV} are individually nitrogen, nitrogen bonded to oxygen, oxygen, sulfur, or carbon bonded to a substituent species characterized as having a sigma m value between about −0.3 and about 0.75; the dotted lines indicate the bonds between adjacent ring atoms may be either single or double bonds; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 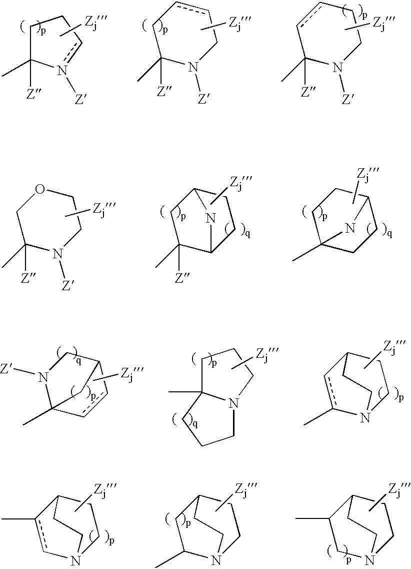
where Z′ individually represent hydrogen or lower alkyl, acyl, alkoxycarbonyl, or aryloxycarbonyl; Z″ is hydrogen or lower alkyl; and Z′″ is a non-hydrogen substituent; the dotted line indicates a carbon-carbon single bond or a carbon-carbon double bond; p is 0, 1 or 2; q is 0, 1, 2 or 3; and j is an integer from 0 to 3.

2. The compound of claim 1 wherein j is 0.

3. The compound of claim 1 wherein p and/or q is 0 or 1.

4. The compound of claim 1 wherein Z′ is hydrogen or methyl, and Z″ is hydrogen.

5. The compound of claim 1 wherein B′ is acetylenic or ethylenic.

6. The compound of claim 1 wherein the two carbon bridging species is —CH═CH—, and that species has an (E) geometry.

7. The compound of claim 1 wherein the two carbon bridging species is ethylenic, and that species has an (E) geometry.

8. The compound of claim 1 wherein all of E, E^{I}, E^{II}, and E^{III} individually are hydrogen.

9. The compound of claim 1 wherein m is 1 and n is 0, and E is hydrogen and E^{I} is methyl.

10. The compound of claim 1 wherein m is 1 and n is 1, and E, E^{I} and E^{II} each are hydrogen and E^{III} is methyl.

11. The compound of claim 1 wherein the sum of m plus n is 1 or 2.

12. The compound of claim 1 wherein one or two of X, X^{I}, X^{II}, X^{III} and X^{IV} are nitrogen or nitrogen bonded to oxygen.

13. The compound of claim 1, wherein the non-hydrogen substituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

14. The compound of claim 1, selected from the group consisting of (E)-5-(2-(5-azabicyclo[3.3.0]octyl)vinyl isoxazole, (E)-5-(2-pyrrolidin-2-ylvinyl isoxazole, (E)-5-(2-(2-piperidyl)vinyl) isoxazole and (E)-5-(2-quinuclidin-2-ylvinyl) isoxazole.

15. A compound of the formula:
Cy—B′—(CEE^{I})ₘ—(CE^{I}E^{III})ₙ—Q
wherein Cy is a 5-membered heteraoromatic ring; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 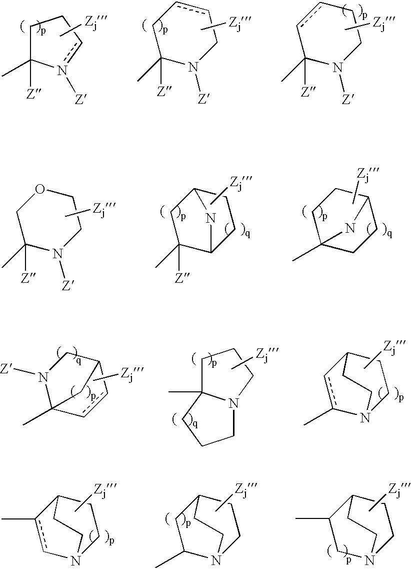
where Z′ individually represent hydrogen or lower alkyl, acyl, alkoxycarbonyl, or aryloxycarbonyl; Z″ is hydrogen or lower alkyl; Z′″ is a non-hydrogen substituent; the dotted line indicates a carbon-carbon single bond or a carbon-carbon double bond; p is 0, 1 or 2; q is 0, 1, 2 or 3; and j is an integer from 0 to 3.

16. The compound of claim 15 wherein j is 0.

17. The compound of claim 15 wherein q is 0 or 1.

18. The compound of claim 15 wherein Z′ is hydrogen or methyl, and Z″ is hydrogen.

19. The compound of claim 15 wherein B′ is acetylenic or ethylenic.

20. The compound of claim 15 wherein the two carbon bridging species is —CH═CH—, and that species has an (E) geometry.

21. The compound of claim 15 wherein the two carbon bridging species is ethylenic, and that species has a an (E) geometry.

22. The compound of claim 15 wherein m and n are 0.

23. The compound of claim 15 wherein m is 1 and n is 0, and E is hydrogen and E^{I} is methyl.

24. The compound of claim 15 wherein m is 1 and n is 1, and E, E^{I} and E^{II} each are hydrogen and E^{III} is methyl.

25. The compound of claim 15 wherein the sum of m plus n is 1 or 2.

26. The compound of claim 15 wherein one or two of X, X^{I}, X^{II}, X^{III} and X^{IV} are nitrogen or nitrogen bonded to oxygen.

27. The compound of claim 15, wherein the non-hydrogen sustituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

28. The compound of claim 15 wherein the 5-membered heteroaromatic ring is selected from the group consisting of isoxazole, isothiazole, oxazole, thiazole, pyrazole, 1,2,4 oxadiazole and 1,2,4-triazole.

29. A pharmaceutical composition incorporating a compound of the formula: 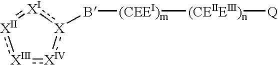
where each of X, X^{I}, X^{II}, X^{III} and X^{IV} are individually nitrogen, nitrogen bonded to oxygen, oxygen, sulfur, or carbon bonded to a substituent species characterized as having a sigma m value between about −0.3 and about 0.75; the dotted lines indicate that bonds between adjacent ring atoms may be either single or double bonds; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 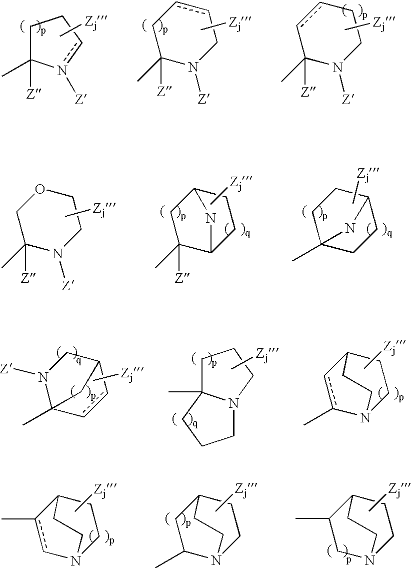
where Z′ individually represent hydrogen or lower alkyl, acyl, alkoxy carbonyl, or aryloxy carbonyl; Z″ id hydrogen or lower alkyl; and Z′″is a non-hydrogen substituent; the do t ted line indicates a carbon-carbon single bond or a carbon-carbon double bond; p is 0, 1 or 2; q is 0, 1, 2 or 3; and j is an integer from 0 to 3.

30. The pharmaceutical composition of claim 29 wherein j is 0.

31. The pharmaceutical composition of claim 29 wherein q is 0 or 1.

32. The pharmaceutical composition of claim 29 wherein Z′ is hydrogen or methyl, and Z″ is hydrogen.

33. The pharmaceutical composition of claim 29 wherein B′ is acetylenic or ethylenic.

34. The pharmaceutical composition of claim 29 wherein the two carbon bridging species is
—CH═CH—, and that species has an (E) geomety.

35. The pharmaceutical composition of claim 29 wherein the two carbon bridging species is ethylenic, and that species has a an (E) geomety.

36. The pharmaceutical composition of claim 29 wherein m and n are 0.

37. The pharmaceutical composition of claim 29 wherein m is 1 and n is 0, and E is hydrogen and E^{I} is methyl.

38. The pharmaceutical composition of claim 29 wherein m is 1 and n is 1, and E, E^{I} and E^{II} each are hydrogen and E^{III} is methyl.

39. The pharmaceutical composition of claim 29 wherein the sum of m plus n is 1 or 2.

40. The pharmaceutical composition of claim 29 wherein one or two of X, X^{I}, X^{II}, X^{III} and X^{IV} are nitrogen or nitrogen bonded to oxygen.

41. The pharmaceutical composition of claim 29, wherein the non-hydrogen substituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

42. The pharmaceutical composition of claim 29, wherein the compound is (E)-5-(2-(5-azabicyclo[3.3.0]octyl)vinyl isoxazole, (E)-5-(2-pyrrolidin-2-ylvinyl isoxazole, (E)-5-(2-(2-piperidyl)vinyl) isoxazole and (E)-5-(2-quinuclidin-2-ylvinyl) isoxazole.

43. A pharmaceutical composition incorporating a compound of the formula:
Cy—B ′—(CEE^{I})ₘ—(CE^{I}E^{III})ₙ—Q
wherein Cy is a 5-membered heteroaromatic ring; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 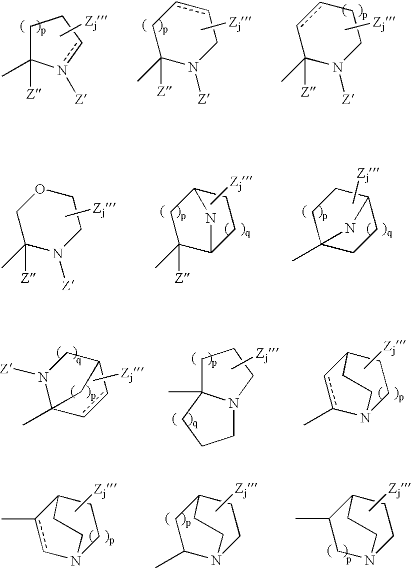
where Z′ individually represent hydrogen or lower alkyl, acyl, alkoxycarbonyl, or aryloxycarbonyl; Z″ is hydrogen or lower alkyl; and Z′″ is a non-hydrogen substituent; the dotted line indicates a carbon-carbon single bond or a carbon-carbon double bond; p is 0, 1 or 2; q is 0, 1, 2 or 3; and j is an integer from 0 to 3.

44. The pharmaceutical composition of claim 43 wherein j is 0.

45. The pharmaceutical composition of claim 43 wherein q is 0 or 1.

46. The pharmaceutical composition of claim 43 wherein Z′ is hydrogen or methyl, and Z″ is hydrogen.

47. The pharmaceutical composition of claim 43 wherein B′ is acetylenic or ethylenic.

48. The pharmaceutical composition of claim 43 wherein the two carbon bridging species is —CH═CH—, and that species has an (E) geometry.

49. The pharmaceutical composition of claim 43 wherein the two carbon bridging species is ethylenic, and that species has a an (E) geometry.

50. The pharmaceutical composition of claim 43 wherein m and n are 0.

51. The pharmaceutical composition of claim 43 wherein m is 1 and n is 0, and E is hydrogen and E^{I} is methyl.

52. The pharmaceutical composition of claim 43 wherein m is 1 and n is 1, and E, E^{I} and E^{II} each are hydrogen and E^{III} is methyl.

53. The pharmaceutical composition of claim 43 wherein the sum of m plus n is 1 or 2.

54. The pharmaceutical composition of claim 43 wherein one or two of X, X^{I}, X^{II}, X^{III} and X^{IV} are nitrogen or nitrogen bonded to oxygen.

55. The pharmaceutical composition of claim 43, wherein the non-hydrogen substituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

56. The pharmaceutical composition of claim 43, wherein the 5-membered heteroaromatic ring is selected from the group consisting of isoxazole, isothiazole, oxazole, thiazole, pyrazole, 1,2,4 oxadiazole and 1,2,4-triazole.

57. A method for treating a central nervous system disorder, said method comprising administering an effective amount of a compound having the formula: 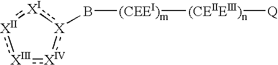
where each of X, X^{I}, X^{II}, X^{III} and X^{IV} are individually nitrogen, nitrogen bonded to oxygen, oxygen, sulfur, or carbon bonded to a substituent species characterized as having a sigma m value between about −0.3 and about 0.75; the dotted lines the bonds between adjacent ring atoms may be either single or double bonds; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 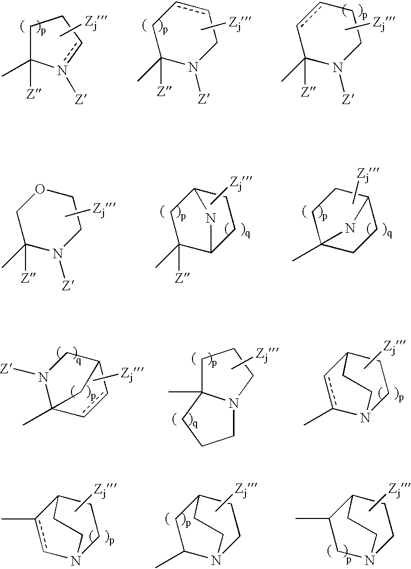
where Z′ individually represent hydrogen or lower alkyl, acyl, alkoxycarbonyl, or aryloxycarbonyl; Z″ is hydrogen or lower alkyl; and Z′″is a non-hydrogen substituent; the dotted line indicates a carbon-carbon single bond or a carbon-carbon double bond; p is 0, 1 or 2; q is 0, 1, 2 or 3; and j is an integer from 0 to 3.

58. The method of claim 57 wherein j is 0.

59. The method of claim 57 wherein q is 0 or 1.

60. The method of claim 57 wherein Z′ is hydrogen or methyl, and Z″ is hydrogen.

61. The method of claim 57 wherein B′ is acetylenic or ethylenic.

62. The method of claim 57 wherein the two carbon bridging species is —CH═CH—, and that species has an (E) geomoetry.

63. The method of claim 57 wherein the two carbon bridging species is ethylenic, and that species has a an (E) geomoetry.

64. The method of claim 57 wherein all of m and n are 0.

65. The method of claim 57 wherein m is 1 and n is 0, and E is hydrogen and E^{I} is methyl.

66. The method of claim 57 wherein m is 1 and n is 1, and E, E^{I} and E^{II} each are hydrogen and E^{III} is methyl.

67. The method of claim 57 wherein the sum of m plus n is 1 or 2.

68. The method of claim 57 wherein one or two of X, X^{I}, X^{II}, X^{III} and X^{IV} are nitrogen or nitrogen bonded to oxygen.

69. The method of claim 57, wherein the non-hydrogen substituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

70. The method of claim 57, wherein the compound is (E)-5-(2-(5-azabicyclo[3.3.0]octyl)vinyl isoxazole, (E)-5-(2-pyrrolidin-2-ylvinyl isoxazole, (E)-5-(2-(2-piperidyl)vinyl) isoxazole and (E)-5-(2-quinuclidin-2-ylvinyl) isoxazole.

71. A method of the formula:
Cy—B′—(CEE^{I})ₘ—(CE^{I}E^{III})ₙ—Q
wherein Cy is a 5-membered heteroaromatic ring; m is an integer and n is an integer such that the sum of m plus n is 0, 1, 2 or 3; B′ is a two carbon bridging species; E, E^{I}, E^{II} and E^{III} individually represent hydrogen or a suitable non-hydrogen substituent; and Q is selected from: 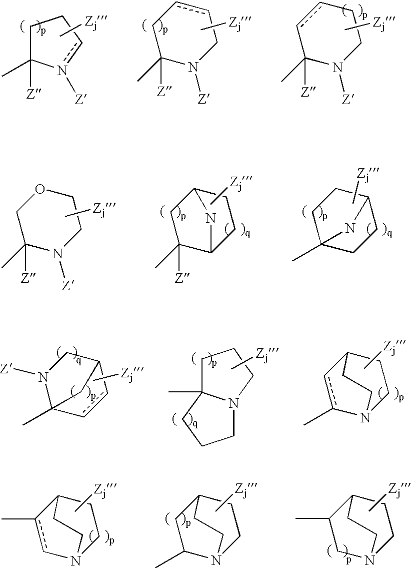
where Z′ and Z″ individually represent hydrogen or lower alkyl, acyl, alkoxycarbonyl, or aryloxycarbonyl; Z′″ is a non-hydrogen substituent; the dotted line indicates a carbon-carbon single bond or a carbon-carbon double bond; p is 0, 1 or 2; q is 0, 1, 2 or 3; and j is an integer from 0 to 3.

72. The method of claim 71 wherein j is 0.

73. The method of claim 71 wherein q is 0 or 1.

74. The method of claim 71 wherein Z′ is hydrogen or methyl, and Z″ is hydrogen.

75. The method of claim 71 wherein B′ is acetylenic or ethylenic.

76. The method of claim 71 wherein the two carbon bridging species is —CH═CH—, and that species has an (E) geometry.

77. The method of claim 71 wherein the two carbon bridging species is ethylenic, and that species has an (E) geometry.

78. The method of claim 71 wherein m and n are 0.

79. The method of claim 71 wherein m is 1 and n is 0, and E is hydrogen and E^{I} is methyl.

80. The method of claim 71 wherein m is 1 and n is 1, and E, E^{I} and E^{II} each are hydrogen and E^{III} is methyl.

81. The method of claim 71 wherein the sum of m plus n is 1 or 2.

82. The method of claim 71 wherein one or two of X, X^{I}, X^{II}, X^{III} and X^{IV} are nitrogen or nitrogen bonded to oxygen.

83. The method of claim 71, wherein the non-hydrogen substituent species is selected from the group consisting of alkyl, substituted alkyl, alkenyl substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, halo, —OR′, —NR′R″, —CF₃, —CN, —NO₂, —C₂R′, —SR′, —N₃, —C(═O)NR′R″, —NR′C(═O) R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)O R″, —SO₂R′, —SO₂NR′R″, and —NR′SO₂R″, where R′ and R″ are individually hydrogen, lower alkyl, cycloalkyl, heterocyclyl, or an aromatic group-containing species and r is an integer from 1 to 6.

84. The method of claim 71 wherein the 5-membered heteroaromatic ring is selected from the group consisting of isoxazole, isothiazole, oxazole, thiazole, pyrazole, 1,2,4 oxadiazole and 1,2,4-triazole.
